(19) United States
(12) Patent Application Publication
(10) Pub. No.: US 2022/0133137 A1
(43) Pub. Date: May 5, 2022

(54) ORGAN FLUSH AND DRAINAGE UNIT

(71) Applicant: Pawel Wisniewski, Wroclaw (PL)

(72) Inventor: Pawel Wisniewski, Wroclaw (PL)

(21) Appl. No.: 17/430,383

(22) PCT Filed: Jan. 29, 2019
(86) PCT No.: PCT/IB2019/050709

§ 371 Date: Aug. 12, 2021

(51) Int. Cl. A61B 1/015; A61B 1/307; A61B 1/00

(52) U.S. Cl. CPC A61B 1/015; A61B 1/00137; A61B 1/00135; A61B 1/307

(57) ABSTRACT

An organ flush and drainage unit for limiting an increase of pressure within a cavity of an organ during liquid irrigation is disclosed. The unit comprises a drainage body enclosing a drainage zone in the form of a drainage chamber as encompassed within a unit wall. The unit also includes a coupling passage in the form of a mouth defined at the opened up end of the chamber. The unit further includes an instrument insertion aperture that is in alignment with the mouth and that is formed with a penetrable sealing means by being fitted with a membrane type penetrable resilient sealing member defining a central location of instrument penetration along which an organ working instrument which is sealably insertable and a drainage cum charging location in the form of a drainage cum charging stub extending from the chamber though the wall of the unit.

### BACKGROUND TO THE INVENTION

The field of endourology includes the drainage of fluid from a kidney during endoscopy in the treatment of renal stones. To maintain clear vision during a procedure of flexible ureterorenoscopy the working area inside a kidney requires proper irrigation while at the same time the maintenance of low pressure during the performance of intrarenal endourological procedures is desirable to limit any kidney damage. It is, amongst others, an objective of this invention to promote an enhanced drainage effect from an upper urinary tract while in the process limiting any build-up of pressure as brought about by the introduction of irrigation liquid through an endoscope to a kidney during the performance of intrarenal procedures. In the field of endourology it is also important to remove small fragments of stones after fragmentation. While the objective of the invention is specifically set out in relation to flexible uretero-endoscopy procedures its application is not necessarily so limited.

### FIELD OF THE INVENTION

This invention relates to organ flush and drainage pressure regulating equipment for at least limiting an increase of pressure within an organ during employment of liquid irrigation in which liquid is supplied into such organ in achieving a medical objective by means of a working instrument that in performing its working action passes along an organ access sheath of which the leading end is positioned into such organ while the trailing end remains outside the body of a patient, and is at a higher elevation than its leading end thereby creating a pressure increasing tendency in an organ. While not so limited the invention finds particular application in the field of kidney endoscopy and the treatment of renal stones.

### DESCRIPTION OF PRIOR ART

It is known to the inventor and applicant that in the prior art an increase in organ pressure in the case of involving a ureteric access sheath (UAS) under circumstances where such pressure increase can manifest has been attempted to be addressed by applying an electronically or mechanically controlled suction to such UAS. See website http://www.ijcem.com/files/ijcem0022325.pdf. As a variation in pressure beyond the normal organ functioning pressure during the performance of a UAS involving medical procedure on an organ such as a kidney can have undesired consequences its proper control is important.

The application of an active suction effect on a UAS can, however, create an over-compensating pressure drop that can have extensively negative consequences for the proper functioning of an organ even leading to, for example, the collapse of a kidney. The present invention overcomes this situation by naturally and thus without the use of specific suction generating equipment to regulate any specific UAS use generated over or under pressure.

### BRIEF DESCRIPTION OF THE DRAWING

The invention is now described, by way of example, with reference to the accompanying drawings. In the drawings

FIG. 1 shows organ flush and drainage pressure regulating equipment in the form of an organ flush and drainage unit in three dimensional view,

FIG. 2 shows the unit in three dimensional sectioned view along section line A-A in FIG. 1,

FIG. 3 shows the unit in side elevation,

FIG. 4 shows the unit in side elevational section along section line B-B in FIG. 5,

FIG. 5 shows the unit in end elevation in the direction of arrow D in FIG. 3

FIG. 6 shows the unit in end elevation in the direction of arrow C in FIG. 3,

FIG. 7 diagrammatically shows the unit as positioned for operative use in conjunction with an organ access sheath in the form of a conventional ureteric access sheath,

FIG. 8 diagrammatically shows the unit as positioned for operative use according to FIG. 7 as holding an organ working instrument in the form a kidney stone removing instrument,

FIG. 9 diagrammatically sets out the functioning of the unit under conditions of patient use,

FIG. 10 gives a detail of the operation of sealing means in the form a membrane type flexible resilient member forming part of the unit in also serving a low pressure relief function in performing a pressure stabilising effect in the case of an excessive pressure change during operative use of the unit, and

FIG. 11 shows the unit in reverse flow application.

### DETAILED DESCRIPTION OF THE DRAWINGS

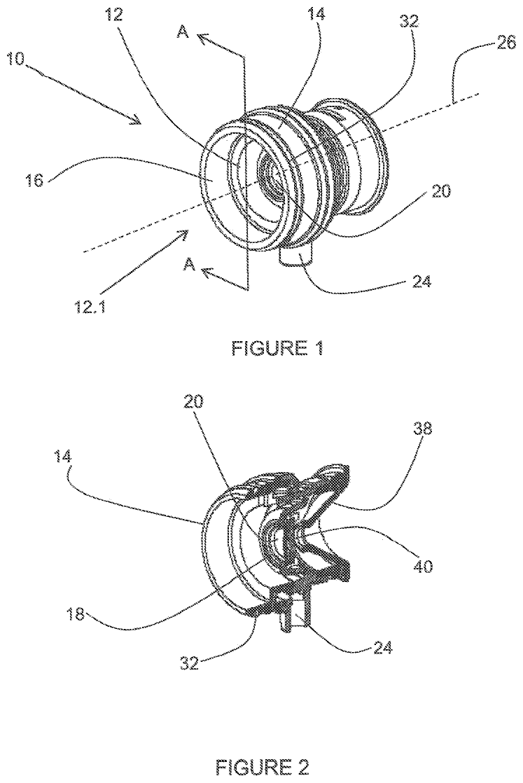

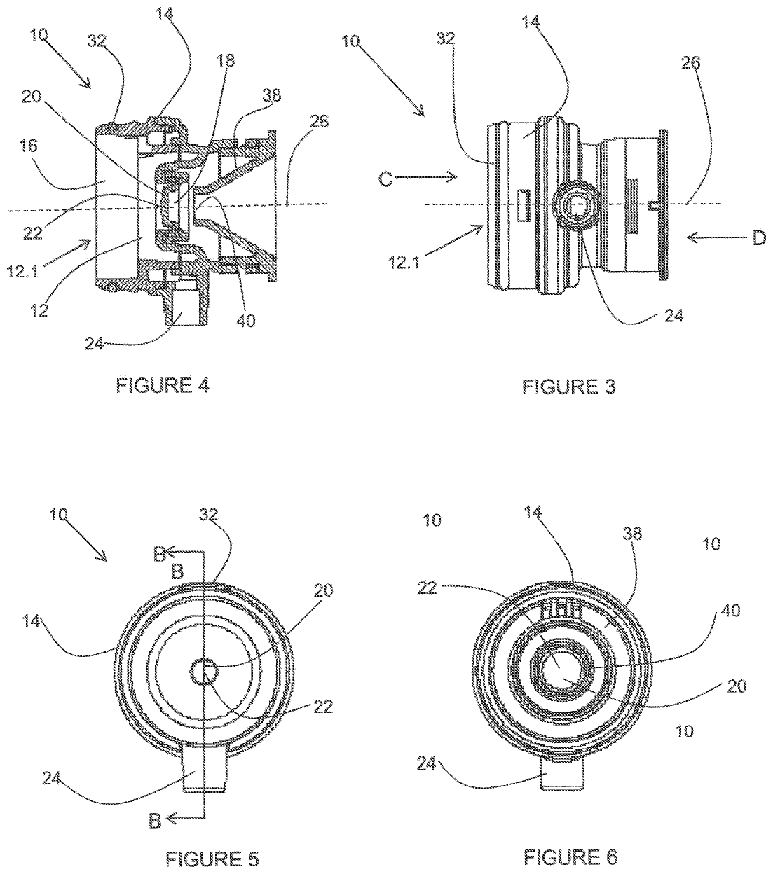

Referring to FIGS. 1 to 8 of the drawings organ flush and drainage pressure regulating equipment in the form of an organ flush and drainage unit for limiting an increase of pressure within a cavity of an organ during liquid irrigation and for performing a low pressure relief effect is generally indicated by reference numeral 10.

The unit comprises a drainage body enclosing a drainage zone in the form of a drainage chamber 12 as encompassed within a unit wall 14, a coupling passage in the form of a mouth 16 defined at the opened up end 12.1 of the chamber 12, an instrument insertion aperture 18 that is in alignment with the mouth 16 and that is formed with penetrable sealing means by being fitted with a membrane type penetrable resilient sealing member 20 defining a central location of instrument penetration 22 along which an organ working instrument which is sealably insertable and a drainage cum charging location in the form of a drainage cum charging stub 24 extending from the chamber 12 though the wall 14 of the unit 10.

The unit 10 extends symmetrically about a central axis 26 as thus passing though the location of instrument penetration 22 along the sealing member 20 and centrally along chamber 12 and mouth 16. The stub 24 extends transversely to the axis 26.

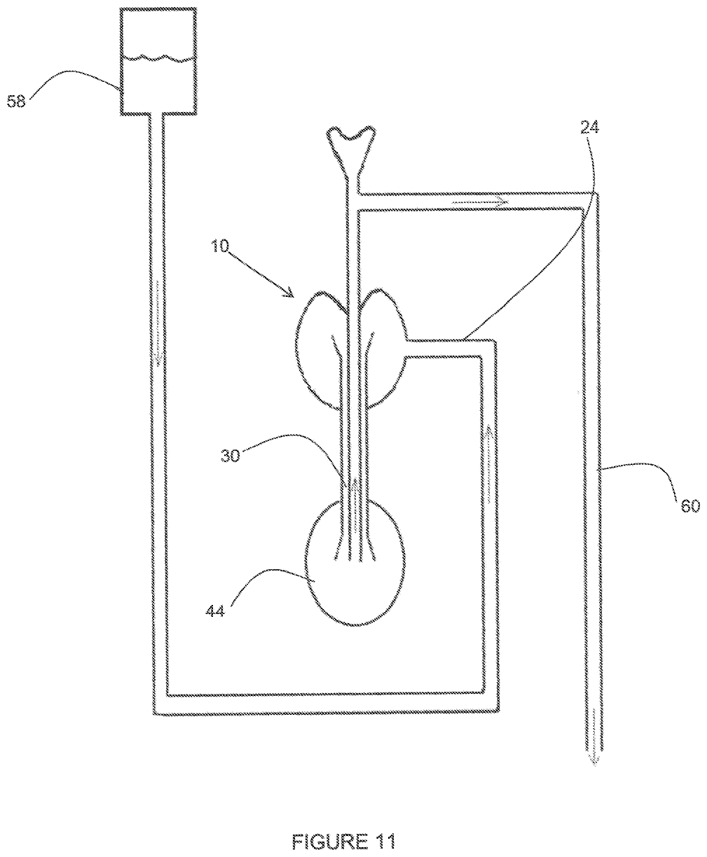

In addition to serving a sealing effect the sealing member 20 also performs a low pressure relief cracking function, as in particular shown in FIG. 10, and an over pressure release function. To this effect and as shown in FIG. 10 the resilience of the member 20 is selected to cause it to become flexibly retracted from its location of instrument penetration 22 into the chamber 12 to such an extent once the pressure in the chamber 12 changes below a sealing member associated minimum value, once the unit 10 is in operative use, to create a path of air ingress enabling the ingress or aspiration of air. The resilience of the member 20 is furthermore selected to flex outward (not shown) once exposed to a pressure beyond a sealing member associated maximum in creating a liquid escape path between the member 20 and an organ working instrument as thus fitted there along in stabilising the chamber associated pressure, once in use. Such action will more often be associated with unit 10 reverse flow application as discussed below with reference to FIG. 11.

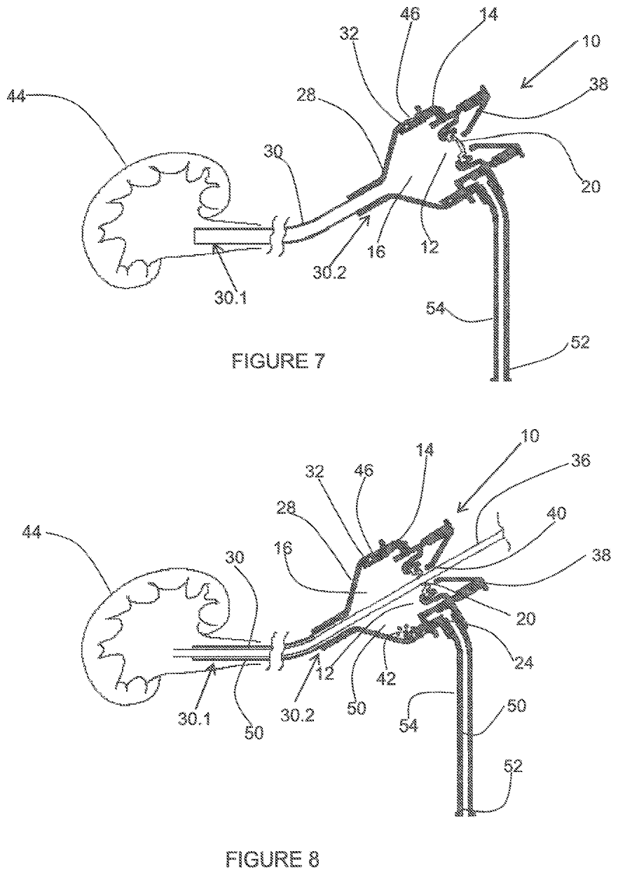

The mouth 16 is sealably engageable against unintentional release to the trailing end of an organ access sheath in the form of a conventional funnel type inlet 28 forming the trailing end 30.2 of a ureteric access sheath 30 by being dimensioned to be socketed into its flared end region 46, as more clearly shown in FIGS. 7 and 8. A sealing effect is attained by way of a circumferential sealing ring 32 extending along the outside face of the wall 14.

To facilitate operative positioning of an organ working instrument such as a kidney stone removing instrument 36 along the unit 10, as is more clearly shown in FIG. 8, it is formed with a guiding formation in the form of a guiding funnel 38 that is integral with the unit 10 and of which the apex 40 is in close proximity to the aperture 18 and thus the location of instrument penetration 22 through member 20.

The organ working instrument as a kidney stone removing instrument 36 or the like often also involves the removal of particulate matter such as kidney (renal) stone remnants. In referring to the FIGS. 8 to 10 and to limit the possibility of particulate matter blocking any outflow along the drainage cum charging stub 24 thus serving as a drainage stub, the latter is positioned to result in its being at a higher elevation than the mouth 16 during operative use of the equipment 10. This enables the collection of particulate matter in the chamber 12 instead of its passing on to the drainage. To this effect the stub 24 extends generally laterally in relation to the axis 26 in being spaced as far as possible from the mouth 16 as permitted by the construction of the unit 10. In being so spaced and referring in particular to FIGS. 7 and 8 in relation to the positioning of unit 10 once operatively used under this embodiment, particulate matter 42 such as renal stones as flushed from an organ along an annular section between the instrument 36 and the ureteric access sheath 30 is caused to gravitationally accumulate within the chamber 12 consequently promoting the free flow of liquid (in the case of kidney stone removal, water) to the stub 24 and onwards for subsequent drainage there from. In the case of an excessive accumulation of particulate matter it will naturally have to be removed from time to time from the chamber 12.

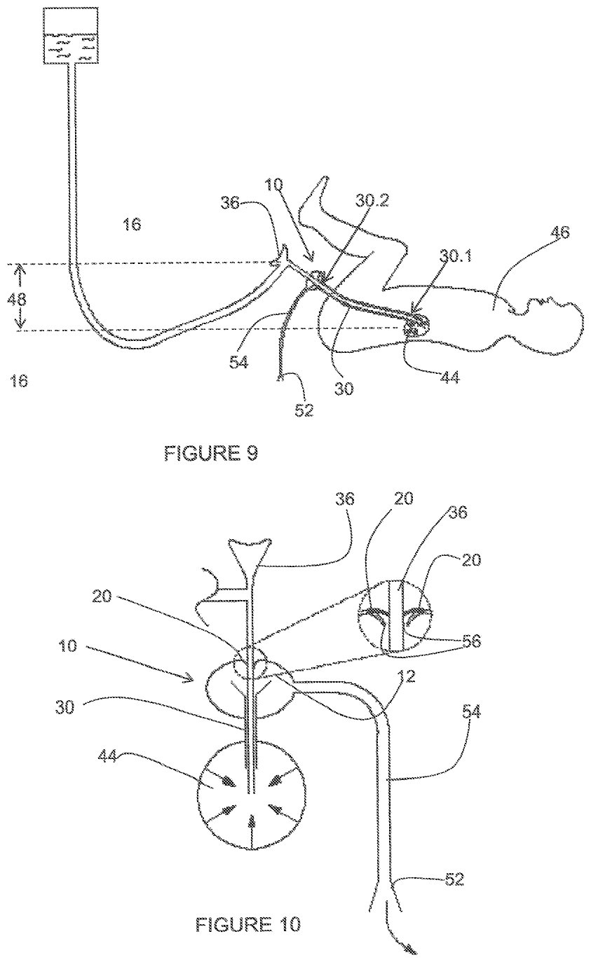

In generally referring to FIG. 9 and in one embodiment of use the performance of a body organ liquid irrigation procedure of the kind in which the unit 10 of the invention finds application such as a renal stone removal procedure, involves the insertion of an organ access sheath such as a ureteric access sheath 30 into the relevant organ 44 such as a kidney with its leading end 30.1 being at a lower elevation that its trailing end 30.2 to attain a proper flushing effect. A patient 46 is under such procedure often positioned in a supine position and in the case of kidney stone removal with lifted legs in lithotomy position as shown in FIG. 9. The sheath 30 thus serves as removal conduit for liquid passed to the organ 44 along an organ working instrument 36 such as a kidney stone removal instrument as conventionally supplied with irrigation liquid to perform its organ application procedure. In the leading end 30.1 of the sheath being at a lower elevation than its trailing end 30.2, increased pressure is exerted on the organ during such procedure as reflected by the elevational spacing 48 reflecting the height of the hydrostatic column of liquid. Such additional pressure is detrimental to the health of the organ consequently requiring mitigation if not removal.

The unit 10 as in use according to the FIGS. 8 and 9 embodiment, forms a sealed flow conduit 50 from the organ 44 along the access sheath 30, though the chamber 12 and up to the stub 24 and beyond. Flushing liquid that passes to the organ access sheath 30 consequently becomes drained via a drainage location 52 at the remote end of a drainage conduit 54 extending from the stub 24. In adjustably selecting the elevation of discharge of the drainage location 52 from a drainage conduit 54, as fitted to the drainage stub 24, below the leading end 30.1 of the organ access sheath 30, an adjustable passive syphon induced suction effect is exerted along the liquid flow path 50. Depending on the elevational location of the drainage location 52 such syphoning effect counteracts if not cancels the increase in supply liquid exerted pressure as discussed above and brought about by the stone removing equipment 36 having the overall effect of limiting if not removing the exertion of flushing liquid exerted pressure on an organ during the performance of an organ involved procedure. And while a liquid flow sealing effect is maintained along the flow conduit 50 during operative use of the unit 10 the functioning of the sealing member 20 ensures the maintenance of the conventional manipulative ability of an organ working instrument 36 as fitted along the unit 10.

In further referring to FIG. 10 and in the sealing member 20 also performing a low pressure relief cracking function, once the pressure along the flow path 50 and in particular in the chamber 12 falls below a sealing member associated minimum, an air flow opening 56 is created between the member 20 an organ working instrument 36 enabling the ingress of air there along until the chamber associated pressure is stabilised again under which condition the sealing member 20 returns to its sealing condition. This function prevents the generation of excessive negative pressures that could possibly be undesirable for the organ. As discussed above an elevated pressure can also be dealt with by the sealing member 20 serving an over pressure relief function in flexing outward to the extent of creating a liquid release path.

As a further application and referring to FIG. 11, the liquid flow through the unit 10 can be reversed and irrigation supplied via the drainage cum charging stub 24 in such case serving liquid supply function as supplied, from an overhead liquid supply source 58. In such case liquid in performing a flushing and fragment removal function flows from the unit 10 down the ureteric access sheath 30, through an organ 44, up the working instrument 30 from where it is discarded along a liquid and fragment drainage 60. In appropriately selecting the length of the drainage 60 a syphoning effect is again achieved with the sealing member 20, in this application again being able to serve a pressure stabilising function as discussed above with specific reference to FIGS. 8 to 10. When used for purely flushing purposes the sealing member 20 will provide a liquid flow over pressure escape path in flexing adequately outward once exposed to a pressure beyond a sealing member associated maximum, in stabilising a chamber associated pressure, once in such use.

It is an advantage of the invention at least as specifically described that it limits if not fully counteracts the build-up of pressure by flushing liquid during endoscopy procedures such as the treatment of renal stones while also accommodating an undesired pressure reduction effect all the time maintaining a proper flushing effect.

1. An organ flush and drainage pressure regulating apparatus for at least limiting an increase of pressure within a body organ during a liquid irrigation procedure in which liquid is flushed there through in achieving a medical objective by means of an organ working instrument that in performing its working action passes along an organ access sheath of which the leading end is positioned into such organ while, once the equipment is in use, a source of supply of irrigation liquid as at a high elevation creates a pressure increasing tendency, the apparatus comprising:
a drainage body enclosing a drainage zone;
a coupling passage extending through a wall of the drainage body that is compatibly dimensioned to enable sealable engagement of the drainage body against unintentional release to the trailing end of such access sheath;
an instrument insertion aperture extending from and in alignment with the coupling passage through the wall of the body to enable the insertion of such organ working instrument there and through the coupling passage along such access sheath once the body is coupled for use; and
a drainage cum charging location along which irrigation liquid flows whether returning along or becoming charged to such access sheath on passing along the drainage zone during an organ working action,
wherein the instrument insertion aperture is formed with a penetrable sealing means that maintains a sealing effect against such organ working instrument once inserted along the instrument insertion aperture against inhibiting its normal operative displacement having the effect, once the equipment is in operative use, of causing a passive, flushing liquid pressure enhancing tendency pressure reducing syphoning action as brought about by the sealing effect of the drainage zone in conjunction with the maintenance of a location of drained liquid release at a generally at least pressure balancing lower elevation than the leading end of such organ access sheath.

2. The apparatus of claim 1, wherein the sealing means is in the form a membrane type penetrable resilient member extending transverse to the direction of insertion of an organ working instrument along the drainage body, and
wherein the membrane type penetrable resilient member is formed with a central location of instrument penetration forming an inner lip at least once penetrated that urges sealably against such organ working instrument once inserted along the resilient member.

3. The apparatus of claim 1, wherein the drainage body is formed with a low pressure relief facility to enable the ingress of environmental air into the drainage zone in performing a pressure stabilising effect in the case of an excessive syphoning pressure reduction effect during operative use of the equipment.

4. The apparatus of claim 3, wherein the low pressure relief facility is incorporated into the sealing means.

5. The apparatus of claim 4, wherein the sealing means is in the form a membrane type penetrable resilient member extending transverse to the direction of insertion of an organ working instrument along the drainage body, and
wherein the membrane type penetrable resilient member is formed with a central location of instrument penetration forming an inner lip at least once penetrated that urges sealably against such organ working instrument once inserted along the resilient member while its resilience is selected to also perform a low pressure relief cracking function once exposed to a pressure of below a sealing member associated minimum.

6. The apparatus of claim 5, wherein the resilience of the sealing member is selected to perform a low pressure relief cracking function once exposed to a pressure of below a sealing member associated minimum in response to its becoming retracted from its inner end into creating an air flow path between the member and an organ working instrument enabling the ingress of air there along until the chamber associated pressure is stabilised again.

7. The apparatus of claim 1, wherein the liquid drainage body is formed with a guiding formation extending away from the sealing means along which an organ working instrument is guidable into and through the instrument insertion aperture once becoming positioned for use.

8. The apparatus of claim 7, wherein the guiding formation is in the form of a guiding funnel that is integrally formed with the liquid drainage body apexing in close vicinity of the instrument insertion aperture.

9. The apparatus of claim 1, wherein the coupling passage extends along a mouth that is sealably engageable with a funnel type inlet forming the trailing end of an organ access sheath in response to being socketably receivable into the flared end region of such inlet in being couplable against unintentional release to the trailing end of such access sheath.

10. The apparatus of claim 9, wherein the mouth is dimensioned to be connected to the inlet funnel of a conventional ureteric access sheath.

11. The apparatus of claim 1, wherein the drainage cum charging location is in the form of a drainage location that performs a drainage function along which irrigation liquid as returning along such access sheath during an organ working action is drained from the drainage zone via the location of drained liquid release of the drainage location.

12. The apparatus of claim 11, wherein the drainage location is situated in an adequately spaced relationship to the coupling passage to cause it to be at an elevated position in relation to the coupling passage during operative use of the apparatus in limiting the ingress of particulate matter thereto in the case of such becoming removed from an organ in response to the operation an organ working instrument also involving such action having the effect of in such matter becoming accumulated within the drainage zone though requiring intermittent removal.

13. The apparatus of claim 1, wherein the drainage cum charging location is in the form of a charging location via which irrigation liquid is charged from a supply source along the coupling passage and an organ access sheath to and through an organ from which it is flushed along an organ working instrument from the remote end region of which a draining location extents that ends in the location of drained liquid release of the drainage location.

14. The apparatus of claim 1, wherein the apparatus is in the form of a unit with the drainage cum charging location being in the form of a drainage cum charging location stub that is connectable by a flexible tube serving either as drainage or charging tube.

15. The apparatus of claim 14, wherein the unit is formed about a central axis extending along the drainage zone, the coupling passage and the instrument insertion aperture as fitted with the sealing means with the drainage cum charging location extending laterally with respect to the axis from the drainage body.

16. The apparatus of claim 15, wherein the drainage zone is in the form of a tubular drainage chamber defined intermediate of the coupling passage and the sealing means.
